(19) United States
(12) Patent Application Publication
(10) Pub. No.: US 2003/0198986 A1
(43) Pub. Date: Oct. 23, 2003

(54) Hippocampus-associated proteins, DNA sequences coding therefor and uses thereof

(75) Inventors: Richard Lathe, Edinburgh (GB); Kenneth Andrew Rose, Edinburgh (GB); Genevieve Stapleton, Seattle, WA (US)

Correspondence Address: NIXON & VANDERHYE, PC, 1100 N GLEBE ROAD, 8TH FLOOR, ARLINGTON, VA, 22201-4714, US

(73) Assignee: BTG International Limited

(21) Appl. No.: 10/373,877

(22) Filed: Feb. 27, 2003

Related U.S. Application Data
(63) Continuation of application No. 09/667,747, filed on Sep. 21, 2000, now abandoned.
Application No. 09/667,747 is a division of application No. 09/270,751, filed on Mar. 17, 1999, now Pat. No. 6,184,350.
Application No. 09/270,751 is a division of application No. 08/845,161, filed on Apr. 21, 1997, now Pat. No. 5,976,850.
Application No. 08/845,161 is a continuation of application No. PCT/GB95/02465, filed on Oct. 18, 1995.

(30) Foreign Application Priority Data
Oct. 19, 1994 (GB) 9421093.7

(51) Int. Cl. C12Q 1/68; C07H 21/04; C12N 9/02; C12P 21/02; C12N 5/06

(52) U.S. Cl. 435/6; 435/69.1; 435/189; 435/320.1; 536/23.2; 435/353

(57) ABSTRACT

This invention provides novel hippocampus-associated proteins and DNA sequences coding therefor. In an investigation of hippocampus-associated proteins by differential screening of a rat hippocampus cDNA library, a cDNA species encoding a novel protein designated Hct-1 was isolated and shown to be a to cytochromes P450. The use of hybridization probes based on the rat Hct-1 sequence has led to the identification of homologues in other mammalian species.

This invention relates to novel hippocampus-associated proteins, to DNA sequences coding therefor, to uses thereof and to antibodies to said proteins. The novel hippocampus-associated proteins are believed to be of the cytochrome P450 class.

### BACKGROUND TO THE INVENTION

The identification of hippocampus-associated proteins and the isolation of cDNA molecules coding therefor is important in the field of neurophysiology. Thus, for example, such proteins are believed to be associated with memory functions and abnormalities in these proteins, including abnormal levels of expression and the formation of modified or mutated protein is considered to be associated with pathological conditions associated with memory impairment. The isolation of novel hippocampus-associated proteins and the associated DNA sequences coding therefor is consequently of considerable importance.

The present invention arose out of our investigation of hippocampus-associated proteins by differential screening of a rat hippocampus cDNA library. A cDNA species encoding a novel protein which we have designated Hct-1 was isolated and shown to be related to cytochromes of the P450 class.

The use of hybridization probes based on the rat Hct-1 sequence has led to the identification of homologues in other mammalian species, specifically mouse and human.

Cytochromes P450 are a diverse group of heme-containing mono-oxygenases (termed CYP's; see Nelson et al., DNA Cell Biol. (1993) 12, 1-51) that catalyse a variety of oxidative conversions, notably of steroids but also of fatty acids and xenobiotics. While CYP's are most abundantly expressed in the testis, ovary, placenta, adrenal and liver, it is becoming clear that the brain is a further site of CYP expression. Several CYP activities or mRNA's have been reported in the nervous system but these are predominantly of types metabolizing fatty acids and xenobiotics (subclasses CYP2C, 2D, 2E and 4). However, primary rat brain-derived glial cells have the capacity to synthesize pregnenolone and progesterone in vitro. Mellon and Deschepper, Brain Res. (1993), 629, 283-292(9) provided molecular evidence for the presence, in brain, of key steroidogenic enzymes CYP11A1 (scc) and CYP11B1 (11β) but failed to detect CYP17 (c17) or CYP11B2 (AS). Although CYP21A1 (c21) activity is reported to be present in brain, authentic CYP21A1 transcripts were not detected in this tissue.

Interest in steroid metabolism in brain has been fuelled by the finding that adrenal- and brain-derived steroids (neurosteroids) can modulate cognitive function and synaptic plasticity. For instance, pregnenolone and steroids derived from it are reported to have memory enhancing effects in mice. However, the full spectrum of steroid metabolizing CYP's in brain and the biological roles of their metabolites in vivo has not been established.

To investigate such regulation of brain function our studies have focused on the hippocampus, a brain region important in learning and memory. Patients with lesions that include the hippocampus display pronounced deficits in the acquisition of new explicit memories while material encoded long prior to lesion can still be accessed normally. In rat, neurotoxic lesions to the hippocampus lead to a pronounced inability to learn a spatial navigation task, such as the water maze. The role of the hippocampus in learning has been further emphasized by the finding that hippocampal synapses, notably those in region CA1, display a particularly robust form of activity-dependent plasticity known as long term potentiation (LTP). This phenomenon satisfies some of the requirements for a molecular mechanism underlying memory processes—persistence, synapse-specificity and associativity. LTP is thought to be initiated by calcium influx through the NMDA (N-methyl D-aspartate) subclass of receptor activated by the excitatory neurotransmitter, L-glutamate, and occlusion of NMDA receptors in vivo with the competitive antagonist AP5 both blocks LTP and the acquisition of the spatial navigation task.

The induction of LTP is attenuated by simultaneous release of gamma-amino butyric acid (GABA) from inhibitory interneurons: activation of GABA_{A} receptors antagonizes L-glutamate induced depolarization of the postsynaptic neuron and interplay between the GABA and L-glutamate receptor pathways is thought to modulate the establishment of LTP. Interplay between these two circuits is emphasised by the finding that some aesthetics (e.g. ketamine) act as antagonists of the NMDA receptor while others, such as the steroid aesthetic alfaxolone, are thought to be agonists of the GABA_{A} receptor. It is of particular note that some naturally occurring steroids, such as pregnenolone sulfate, act as agonists of the GABA_{A} receptor, while pregnenolone sulfate is also reported to increase NMDA currents. Although neurosteroids principally appear to exert their effects via the GABA_{A} and NMDA receptors, there have been indications that neurosteroids may also interact with sigma and progesterone receptors.

Despite considerable interest in the action of neuro-active steroids, and possible roles in modulating synaptic plasticity and brain function, little is known of pathways of steroid metabolism in the central nervous system. As part of a study into the molecular biology of the hippocampal formation, and the mechanisms underlying synaptic plasticity, we have sought molecular clones corresponding to mRNA's expressed selectively in the formation. One such cDNA, Hct-1 (for hippocampal transcript), was isolated from a cDNA library prepared from adult rat hippocampus. Sequence analysis has revealed that Hct-1 is a novel cytochrome P450 most closely related to cholesterol- and steroid-metabolizing CYP's but, unlike other CYP's, is predominantly expressed in brain. The present invention provides molecular characterization of Hct-1 coding sequences from rat, mouse and humans, their expression patterns, and discusses the possible role of Hct-1 in steroid metabolism in the central nervous system.

DNA sequences encoding hitherto unknown cytochrome P450 proteins have now been identified and form one aspect of the present invention.

### SUMMARY OF THE INVENTION

According to one aspect of the present invention there are thus provided DNA molecules selected from the following:

(a) DNA molecules containing the coding sequence set forth in SEQ Id No: 1 beginning at nucleotide 22 and ending at nucleotide 1541,

(b) DNA molecules containing the coding sequence set forth in SEQ Id No: 2 beginning at nucleotide 1 and ending at nucleotide 1242,

(c) DNA molecules capable of hybridizing with the DNA molecule defined in (a) or (b) under standard hybridization conditions defined as 2×SSC at 65° C.

(d) cytochrome P450-encoding DNA molecules capable of hybridizing with the DNA molecule defined in (a), (b) or (c) under reduced stringency hybridization conditions defined as 6×SSC at 55° C.

Such DNA sequences can represent coding sequences of Hct-1 proteins. The sequences (a) and (b) above represent the mouse and rat Hct-1 gene sequence. Homologous sequences from other vertebrate species, especially mammalian species (including man) fall within the class of DNA molecules represented by (c) or (d).

Thus the present invention further provides a DNA molecule consisting of sequences of the human Hct-1 gene.

These DNA sequences may be selected from the following:

(e) DNA molecules comprising one or more sequences selected from

(i) the sequence designated “intron 2” in SEQ Id No 3,

(ii) the sequence designated “exon 3” in SEQ Id No 3,

(iii) the sequence designated “intron 3” in SEQ Id No 3,

(iv) the sequence designated “exon 4” in SEQ Id No 3, and

(v) the sequence designated “intron 5” in SEQ Id No 3; and

(f) DNA molecules capable of hybridizing with the DNA molecules defined in (e) under standard hybridization conditions defined as 2×SSC at 65° C.

(g) cytochrome P450-encoding DNA molecules capable of hybridizing with the DNA molecule defined in (e) or (f) under reduced stringency hybridization conditions defined as 6×SSC at 55° C.

(h) DNA molecules comprising contiguous pairs of sequences selected from

(i) the sequence designated “intron 2” in SEQ Id No 3,

(ii) the sequence designated “exon 3” in SEQ Id No 3,

(iii) the sequence designated “intron 3” in SEQ Id No 3,

(iv) the sequence designated “exon 4” in SEQ Id No 3, and

(v) the sequence designated “intron 5” in SEQ Id No 3; and

(i) DNA molecules capable of hybridizing with the DNA molecules defined in (h) under standard hybridization conditions defined as 2×SSC at 65° C.

(j) cytochrome P450-encoding DNA molecules capable of hybridizing with the DNA molecule defined in (h) or (i) under reduced stringency hybridization conditions defined as 6×SSC at 55° C.

(k) DNA molecules comprising a contiguous coding sequence consisting of the sequences “exon 3” and “exon 4” in SEQ Id No 3, and

(l) DNA molecules capable of hybridizing with the DNA molecules defined in (k) under standard hybridization conditions defined as 2×SSC at 65° C.

(m) cytochrome P450-encoding DNA molecules capable of hybridizing with the DNA molecule defined in (k) or (I) under reduced stringency hybridization conditions defined as 6×SSC at 55° C.

It will be appreciated that the DNA sequences that include introns (such as the sequences covered by definitions (e) to (j) above), may consist of or be derived from genomic DNA. Those sequences that exclude introns may also be genomic in origin, but typically would consist of or be or be derived from cDNA. Such sequences could be obtained by probing an appropriate library (cDNA or genomic) using hybridisation probes based upon the sequences provided according to the invention, or they could be prepared by chemical synthesis or by ligation of sub-sequences.

The invention further provides DNA molecules encoding an Hct-1 gene-associated sequence coded for by a DNA molecule as defined above, but which differ in sequence from said sequences by virtue of one or more amino acids of said Hct-1 gene-associated sequences being encoded by degenerate codons.

The present invention further provide DNA molecules useful as hybridization probes and consisting of a contiguous sequence of at least 18 nucleotides from the DNA sequence set forth in SEQ Id Nos: 1, 2 and 3.

Such molecules preferably contain at least 24 and more preferably at least 30 nucleotide taken from said sequences.

The aforementioned DNA molecules are useful as hybridization probes for isolating members of gene families and homologous DNA sequences from different species. Thus, for example, a DNA sequence isolated from one rodent species, for example rat, has been used for isolating homologous sequences from another rodent species, for example mouse and from other mammalian species , e.g. primate species such as humans.

Such sequences may be further used for isolating homologous sequences from other mammalian species, for example domestic animals such as cows, horses, sheep and pigs, primates such as chimpanzees, baboons and gibbons.

DNA sequences according to the invention may be used in diagnosis of neuropsychiatric disorders, endocrine disorders, immunological disorders, diseases of cognitive function, neurodegenerative diseases or diseases of cognitive function, for example by assessing the presence of depleted levels of mRNA and/or the presence of mutant or modified DNA molecules. Such sequences include hybridisation probes and PCR primers. The latter generally would be short (e.g. 10 to 25) oligonucleotides in length and would be, capable of hybridising with a DNA molecule as defined above. The invention includes the use of such primers in the detection of genomic or cDNA from a biological sample for the purpose of diagnosis of neuropsychiatric disorders, endocrine disorders, immunological disorders, diseases of cognitive function or neurodegenerative diseases.

The present invention further provides hippocampus-associated proteins as such, encoded by the DNA molecules of the invention.

In particular, there is provided

(i) the protein designated rat Hct-1 comprising the amino acid sequence set forth in SEQ Id No: 1 or a protein having substantial homology thereto,

(ii) the protein designated mouse Hct-1 comprising the amino acid sequence set forth in SEQ Id No: 2 or a protein having substantial homology thereto, or

(iii) the protein designated human Hct-1 comprising the amino acid sequence set forth in SEQ Id No: 3 or a protein having substantial homology thereto.

By “substantial homology” is meant a degree of homology such that at least 50%, preferably at least 60% and most preferably at least 70% of the amino acids match. The invention of course covers related proteins having a higher degree of homology, e.g. at least 80%, at least 90% or more.

The Hct-1 polypeptides may be produced in accordance with the invention by culturing a transformed host and recovering the desired Hct-1 polypeptide, characterised in that the host is transformed with nucleic acid comprising a coding sequence as defined above.

Examples of suitable hosts include yeast, bacterial, insect or mammalian cells. Although vectorless expression may be employed, it is preferred that the nucleic acid used to effect the transformation comprises an expression construct or an expression vector, e.g. a vaccinia virus, a baculovirus vector, a yeast plasmid or integration vector.

The invention further provides antibodies, especially monoclonal antibodies which bind to Hct-1 proteins. These and the proteins of the invention may be employed in the design and/or manufacture of an antagonist to Hct-1 protein for diagnosis and/or treatment of diseases of cognitive function or neurodegenerate diseases. The use of Hct-1-associated promoters in the formation of constructs for use in the creation of transgenic animals is also envisaged according to the invention. The antibodies of the invention may be prepared in conventional manner, i.e. by immunising animal such as rodents or rabbits with purified protein obtained from recombinant yeast, or by immunising with recombinant vaccinia.

Hct-1 proteins provided according to the invention posseses catalytic activity, thus they may be used in industrial processes, to effect a catalytic transformation of a substrate. For example, where the substrate is a steroid, the proteins may be used to catalyse stereospecific transformations, e.g. transformations involving oxygen transfer.

### DESCRIPTION OF DRAWINGS (SEE ALSO FIGURE LEGENDS—7 INFRA)

FIG. 1 illustrates (a) a restriction map of clone 12 and (b) the complete nucleotide and translation sequence of the 1.4 kb cDNA clone of rat Hct-1,

FIG. 2 illustrates Northern analysis of Hct-1 expression in adult rat and mouse brain, and other tissues,

FIG. 3 illustrates (a) restriction maps of clones 35 and 40 and (b) the complete nucleotide and translation sequence of mouse Hct-1 cDNA,

FIG. 4 illustrates an alignment of mouse Hct-1 with human CYP7 and highlights regions homologous to other steroidogenic P450s,

FIG. 5 illustrates an analysis of Hct-1 expression in mouse brain,

FIG. 6 illustrates Southern analysis of Hct-1 coding sequences in mouse, rat and human.

FIG. 7 illustrates Southern blot analyses of mouse genomic DNA using (a) a full length mouse Hct-lcDNA clone and (b) rat genomic DNA probed with clone 14.5a,

FIG. 8 illustrates a genomic map of mouse Hct-1,

FIG. 9 illustrates a partial nucleotide sequence of human genomic Hct-1 (CYP7B1) and the encoded polypeptide,

FIG. 10 illustrates an amino acid alignment of mouse Hct-1 and human CYP7,

FIG. 11A illustrates Kozak sequences in mRNAs for steroidogenic P540's,

FIG. 11B illustrates mutagenesis of the 5′ end of the mouse Hct-1 cDNA to sreate a near-consensus translation initiation region surrounding the ATG (AUG),

FIG. 12 illustrates yeast expression vectors containing the mouse Hct-1 coding sequence, and

FIG. 13 illustrates a vaccinia expression vectors containing the mouse Hct-1 coding sequence.

### DESCRIPTION OF SPECIFIC EMBODIMENTS

Details of the isolation of hippocampus-associated DNA molecules according to the invention will now be described by way of example:

1. Isolation of Gene Encoding Rat HCT-1

1.1 Differential Screening of a Rat Hippocampus cDNA Library

To identify genes whose expression is enriched in the hippocampal formation we performed a differential hybridization screen of a hippocampal cDNA library. Adult rat hippocampal RNA was reverse transcribed using a oligo-dT-NotI primer, converted to double-stranded cDNA, EcoRI adaptors were attached and the cDNA's were inserted between the EcoRI and NotI sites of a bacteriophage lamda vector.

1.1.1 Preparation of cDNA Libraries

Following anaesthesia (sodium pentobarbital) of adult rats (Lister hooded) the hippocampal formation was dissected, including areas CA1-3 and dentate gyrus, subiculum, alvear and fimbrial fibres but excluding fornix and afferent structures such as septum and entorhinal cortex. Remainder of brain was also pooled taking care to exclude hippocampal tissue. Total RNAs were prepared by a standard guanidinium isothiocyanate procedure, centrifugation through a CsCl cushion, and poly-A⁺ mRNA selected by affinity chromatography on oligo-dT cellulose. First strand cDNA synthesis used a NctI adaptor primer

[5-dCAATTCGCGGCCGC(T)₁₅-3′]

and Moloney murine leukemia virus (MMLV) reverse transcriptase; second strand synthesis was performed by RNaseH treatment, DNA polymerase I fill-in and ligase treatment. Following the addition of hemi-phosphorylated EcoRI adaptors (5′-dCGACAGCAACGG-3′ and 5′-dAATTCCGTTGCTGTCG-3′) and cleavage with NotI the cDNA was inserted between the Noti and EcoRI sites of bacteriophage lambda vector lambda-ZAPII (Stratagene).

1.1.2 Differential Hybridization Screening

Recombinant bacteriophage plaques were transferred in duplicate to Hybond-N membranes (Amersham), denatured (0.5 M NaOH, 1.5 M NaCl, 4 min), renatured (1 M Tris.HCl pH 7.4, 1.5 M NaCl), rinsed, dried and baked (2 h, 80° C.). Hybridization as described (Church et al., Proc. Natl. Acad. Sci. USA (1984), 81 1991-1995) used a radiolabelled probe prepared by MMLV reverse transcriptase copying of polyA⁻ RNA (from either hippocampus or the remainder of brain) into cDNA in the presence of α-³²P-dCTP and unlabelled dGTP, dATP and dTTP according to standard procedures. Following washing and exposure for autoradiography, differentially hybridizing plaques were repurified. Inserts were transferred to a pBluescript vector either by cleavage and ligation or by using in vivo excision using the ExAssist/SOLR system (Stratagene).

Duplicate lifts from 500,000 plaques were screened with radiolabelled cDNA probes prepared by reverse transcription of RNA from either hippocampus (Hi) or ‘rest of brain’ (RB). Approximately 360 clones gave a substantially stronger hybridization signal with the Hi probe than with the RB probe; 49 were analysed in more depth. In vivo excision was used to transfer the inserts to a plasmid vector for partial DNA sequence studies. Of these, 21 were novel (not presented here); others were known genes whose expression is enriched in hippocampus but not specific to the formation (eg., the rat amyloidogenic protein. Northern analysis was first performed using radiolabelled probes corresponding to the 21 novel sequences. While three (12.10a, 14.5a and 15.13a) identified transcripts specific to the hippocampus, 12.10a and 15.13a both hybridized to additional transcripts whose expression was not restricted to the formation. Clone 14.5a appeared to identify transcripts enriched in hippocampus and was dubbed Hct-1.

1.2 Characterisation of Rat Hct-1

1.2.1 Rat Hct-1 Encodes a Cytochrome P450

To extend this characterization, the insert of clone 14.5a (300 nt) was used to rescreen the hippocampal cDNA library. 4 positives were identified (clones 14.5a-5, −7, −12 and −13), and the region adjacent to the poly-A tail analysed by DNA sequencing. While clones 5 (0.7 kb) and 12 (1.4 kb) had the same 3′ end as the parental clone, clone 7 (0.9 kb) had a different 3′ end consistent with utilization of an alternative polyadenylation site. Clone 13 (2.5 kb), however, appeared unrelated to Hct-1 and was dubbed Hct-2.

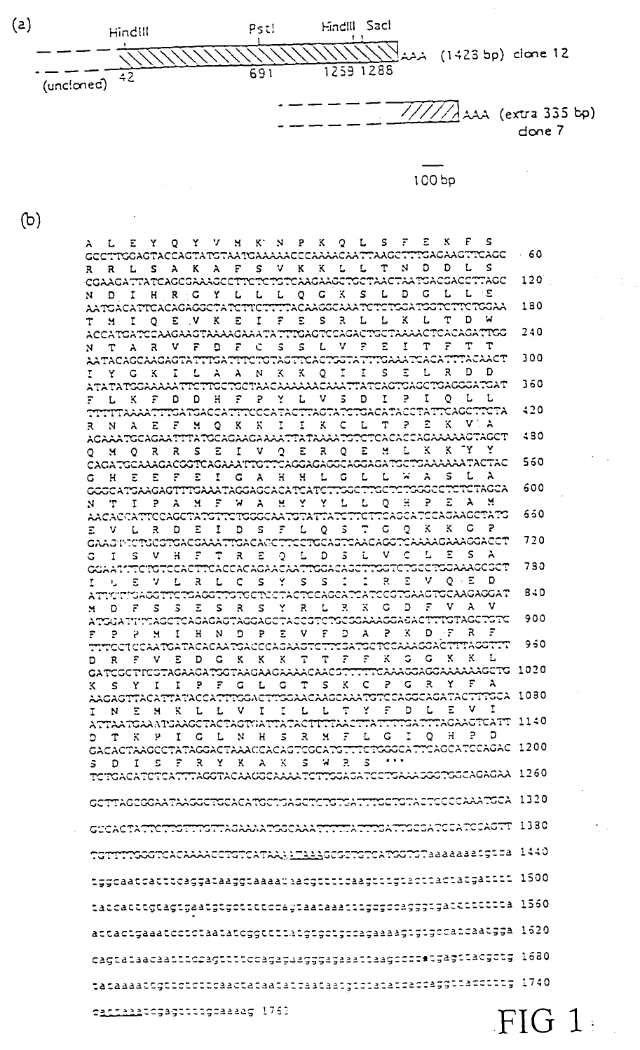

Clones 12 and 7 were then fully sequenced and the sequences obtained were compared with the database. Significant homology was detected between clone 12 and the human and rat cDNA's encoding cholesterol 7α-hydroxylase, though the sequences are clearly distinct. At the nucleic acid level, the 1428 nt cDNA clone for rat Hct-1 shared 55% identity over an 1100 nt overlap with human cholesterol 7α-hydroxylase (CYP7) and 54% identity over a 1117 nt overlap with rat CYP7. FIG. 1 gives the partial cDNA sequences of rat Hct-1 and the encoded polypeptide.

1.2.2 Nct-1 mRNA Expression in Rat

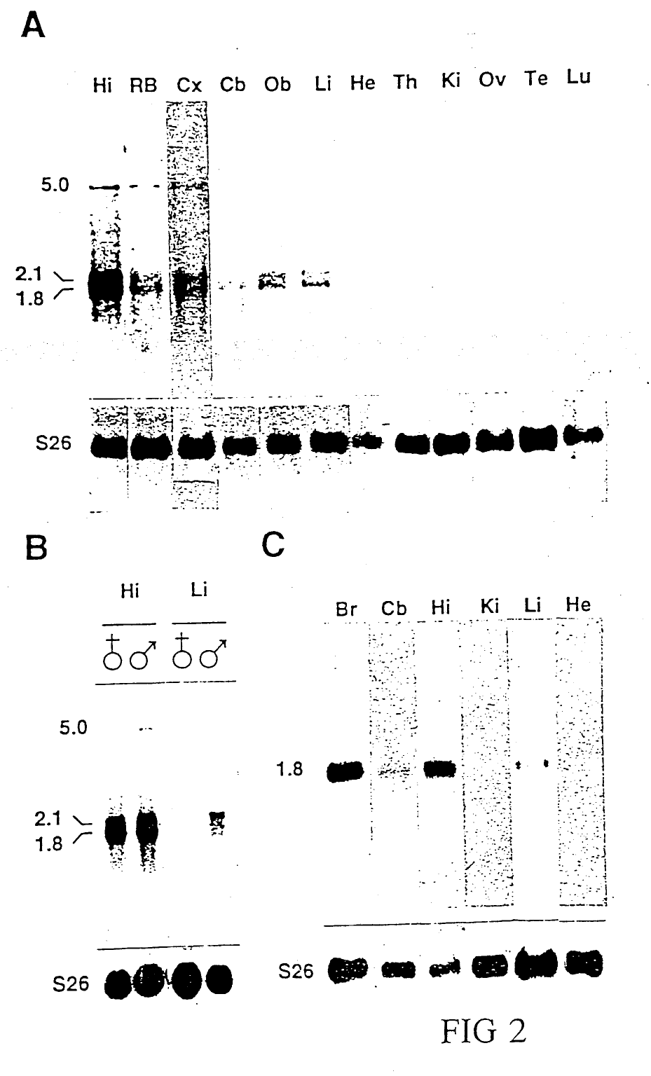

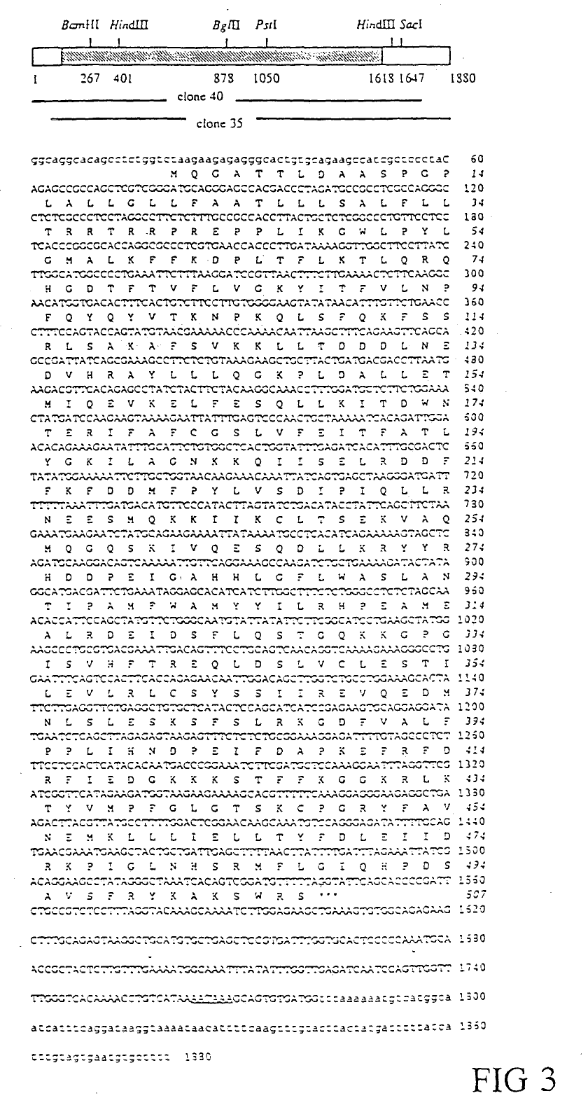

Rat Hct-1 clone 14.5a/12 (1.4 kb) was used to investigate the expression of Hct-1 mRNA in rat brain and other organs. We first performed in situ hybridization to sections of rat brain. While these preliminary experiments did not permit unambiguous localization of Hct-1 transcripts, we confirmed expression in the hippocampus, predominantly in the cell layers of the dentate gyrus, while weaker expression was detected in other hippocampal and brain regions (not presented). Northern analysis was then performed on RNA prepared from different sections of rat brain. In FIG. 2A the Hct-1 probe identifies three transcripts in hippocampus of 5.0, 2.1 and 1.8 kb, with the two smaller transcripts being particularly enriched in hippocampus. The larger transcript was only detectable in brain, while the two smaller transcripts were also present in liver (and, at much lower levels, in kidney) but not in other organs tested including adrenal (not shown), testis, and ovary. In brain, expression was also detected in olfactory bulb and cortex while very low levels were present in cerebellum (FIG. 2A).

1.2.3 Sexual Dimorphism of Hct-1 Expression in Liver but not in Brain

The expression of several CYPS is known to be sexually dimorphic in liver. We therefore inspected liver and brain of male and female rats for the presence of Hct-1 transcripts. In FIG. 2B the Hct-1 probe revealed the 1.8 and 2.1 kb (and 5.0 kb, Hct-2) transcripts in both male and female brain, with the 2.1 kb Hct-1 transcript predominating. Levels of Hct-1 mRNA's in liver were reduced greater than 20-fold over those detected in brain. Furthermore, Hct-1 transcripts were only significant in liver from male animals; expression of Hct-1 in females was barely detectable demonstrating that hepatic expression of Hct-1 is sexually dimorphic.

2. Isolation of Mouse HCT-1

2.1 Isolation of Mouse Hct-1 cDNA Clones

A mouse liver cDNA library, established as Notl-EcoRi fragments in a lambda-gt10 vector, was probed using a rat Hct-1 probe. The library was a kind gift of B. Luckow and K. Kästner, Heidelberg.

Because the transcripts identified by the Hct-1 probe (predominantly 1.8 and 2.1 kb) are clearly longer than the longest cDNA clone (1.4 kb) obtained from our rat hippocampus library, we therefore elected to pursue studies with the mouse Hct-1 ortholog. A mouse liver cDNA library was screened using a rat Hct-1 probe and four clones were selected, none containing a poly-A tail. Two (clones 33 and 35, both 1.8 kb) gave identical DNA sequences at both their 5′ and 3′ ends, and this sequence was approximately 91% similar to rat Hct-1. The remaining two clones, 23 and 40, were also identical to each other and were related to the other clones except for a 5′ extension in (59 nt) and a 3′ deletion (99 nt). The complete DNA sequences of clones 35 and 40 were therefore determined.

The sequences obtained were identical throughout the region of overlap. The mouse Hct-1 open reading frame (ORF) commences with a methionine at nucleotide 81 (numbering from clone 40) and terminates with a TGA codon at nucleotide 1600, encoding a protein of 507 amino acids (FIG. 3). At the 5′ end it is of note that the ATG initiation codon leading the ORF does not correspond to the translation initiation consensus sequence YYAYYATGR. However, the 5′ untranslated region cloned is devoid of other possible initiation codons and an in-frame termination triplet (TAA) lies 20 codons upstream of the ATG. The encoded polypeptide sequence aligns well with other cytochrome P450 sequences and we surmise that the ATG at position 81 represents the correct start site for translation. At the 3′ end the truncation of clone 40 lies entirely in the non-coding region downstream of the stop codon. Neither clone contained a poly-A tail but both contained a potential polyadenylation sequence (AATAAA) at a position corresponding precisely to that seen in the rat cDNA.

2.2 Structure of Mouse Hct-1 Polypeptide

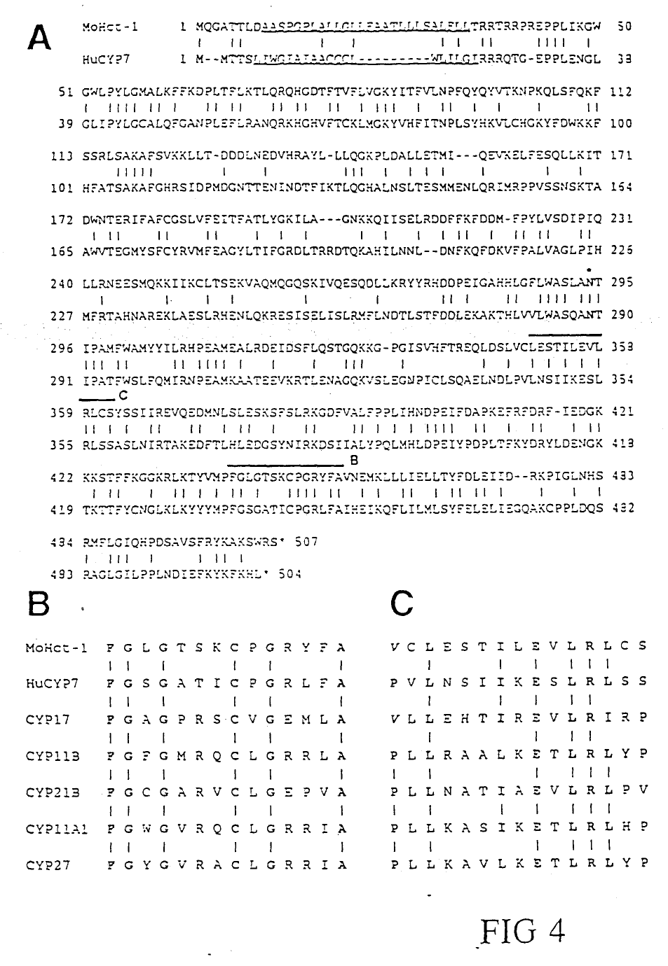

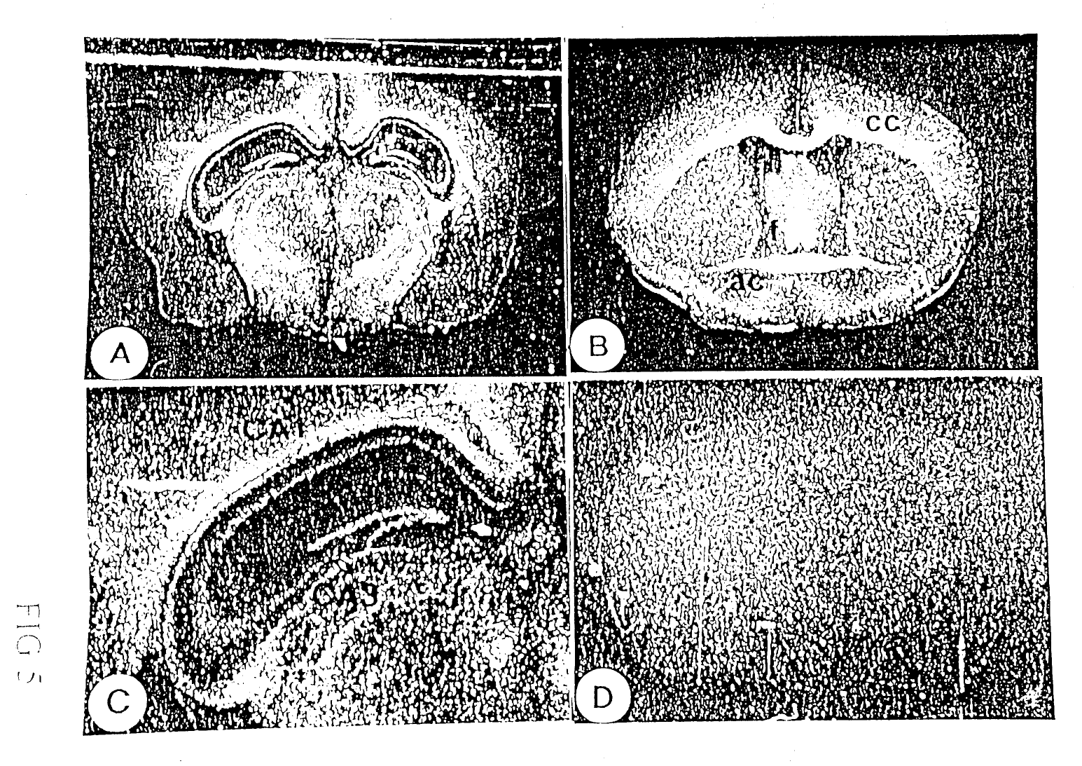

As anticipated, nucleotide sequence homology of mouse Hct-1 was highest with human cholesterol 7α-hydroxylase, with approximately 56% identity over the coding region. At the polypeptide level the mouse ORF shows 81% identity to the rat Hct-1 polypeptide over 414 amino acids; the precise degree of similarity may be different as the full protein sequence of rat Hct-1 is not known. Both the human (CYP7) and rat cholesterol 7α-hydroxylase polypeptides share 39% amino acid sequence identity to mouse Hct-1. FIG. 4A presents the alignment of mouse Hct-1 polypeptide with human CYP7.

The N-terminus of the Hct-1 polypeptide is hydrophobic, a feature shared by microsomal CYP's. This portion of the polypeptide is thought to insert into the membrane of the endoplasmic reticulum, holding the main bulk of the protein on the cytoplasmic side. Consistent with microsomal CYP's, the N-terminus lacks basic amino acids prior to the hydrophobic core (amino acids 9-34).

Several alignment studies have previously highlighted conserved regions within CYP polypeptides. We therefore inspected the Hct-1 sequence for these conserved regions. CYP's contain a highly conserved motif, FxxGxxxCxG(xxxA), present in 202 of the 205 compiled sequences (Nelson et al., supra), that is thought to represent the heme binding site. The arrangement of amino acids around the cysteine residue has been postulated to preserve the three-dimensional structure of this region for ligand binding. This motif is fully conserved in Hct-1 (FIG. 4B). A second conserved domain is also present in CYP's responsible for steroid interconversions. While this domain is largely conserved in Hct-1 an invariant Pro residue is replaced, in Hct-1, by Val (FIG. 4C); the rat Hct-1 polypeptide also contains a Val residue at this position.

2.3 Expression Pattern of Mouse Hct-1

To verify enriched expression of Hct-1 in hippocampus we performed Northern and in situ hybridization analyses on mouse material. In contrast to the situation in rat, the 1.4 kb clone 12 detected only a 1.8 kb transcript; the 2.1 kb and 5.0 kb transcripts were absent from all tissues examined (FIG. 2C). The apparent absence of the 2.1 kb transcript may only reflect a lower abundance of this transcript. because at least some mouse cDNA clones extend beyond the upstream polyadenylation site which is thought, in rat, to generate the shorter (1.8 kb) transcript.

To refine this analysis, a 42-mer oiigonucleotide was designed according to the DNA sequence of the 3′ untranslated region of the cDNA clone upstream of the first polyadenylation site (materials and methods), so as to minimize cross-hybridization with other CYP mRNA's. Coronal sections of mouse brain were hybridized to the ³⁵S-labelled probe and, after emulsion dipping, exposed for autoradiography (FIG. 5). Transcripts were detected throughout mouse brain, with no evidence of restricted expression in the hippocampus (FIG. 5A,B). Strongest expression was observed in the corpus callosum, the anterior commisure and fornix while, as in rat, hippocampal expression was particularly prominent in the dentate gyrus (FIG. 5C). Moderate expression levels, comparable to those observed in hippocampus, were observed in cerebellum, cortex and olfactory bulb.

2.4 The structure of the mHct-1 Gene.

The use of homologous recombination to manipulate the mouse Hct-1 gene requires knowledge of the intron-exon structure of the gene. Sequences upstream of the first Hct-1 exon could also be analysed for elements which contribute to the transcriptional regulation of Hct-1 expression. For these reasons, the organisation of the mouse Hct-1 gene was investigated.

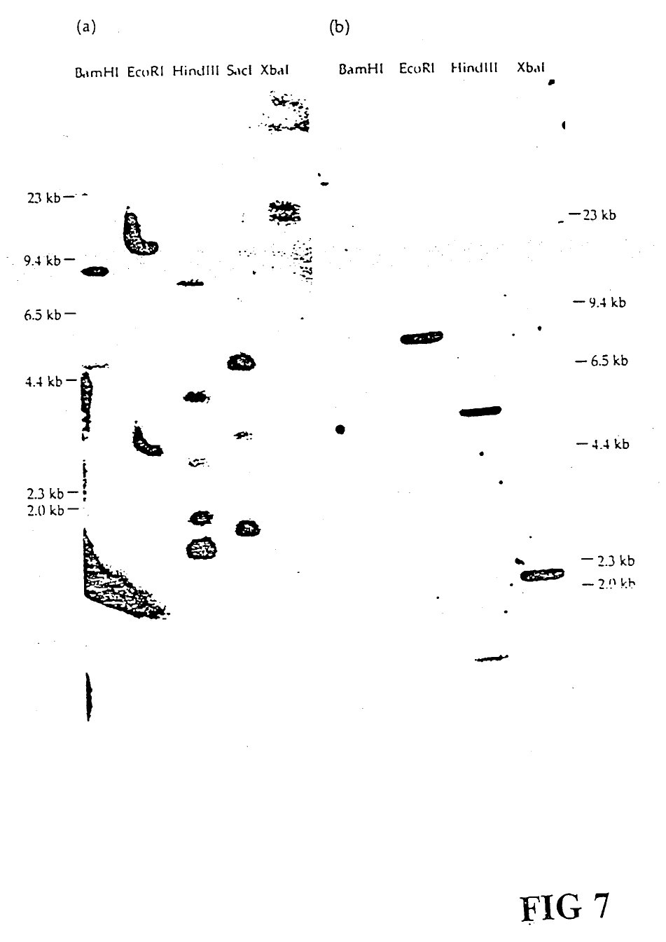

To assess the complexity of the Hct-1 gene in the genome, that is, whether the Hct-1 gene is present as a single copy in the haploid mouse genome, and to assist in mapping of mHct-1 phage clones, the 1.8 kb full length mouse Hct-1 clone was ³²P-labelled by random primer labelling and used as a probe on a Southern blot of mouse genomic DNA (FIG. 7(a)). Under high stringency conditions the Hct-1 probe recognised a small number of bands within the mouse genomic digests, suggesting that Hct-1 is present in the mouse genome as a single copy gene. To confirm this, the original 0.3 kb cDNA clone, 14.5a, was used to probe a rat genomic Southern blot. The smaller probe hybridised to a single band in BamHI-, EcoRI-, and XbaI-digested genomic rat DNA (FIG. 7(b)).

A mouse genomic DNA library (a gift from A. Reaume, Toronto) prepared from ES cells derived from the 129 mouse strain was screened for genomic clones containing mHct-1 exonic sequence. 750,000 recombinant phage of the lambda DASH II library were plated at a density of 50,000 recombinants per 15 cm plate. Duplicate lifts were made and probed with the 1.4 kb rat Hct-1 clone. After the primary screen, 5 clones were isolated. After secondary screening, three of these phage clones were positive and were purified.

Small scale phage DNA was prepared from each phage lysate and cut with NotI to release the inserts. No internal NotI sites were found in any of the clones. Clone I-2 contained a 14 kb insert; clone I-6 contained a 15 kb insert, and clone I-11 contained a 12 kb insert.

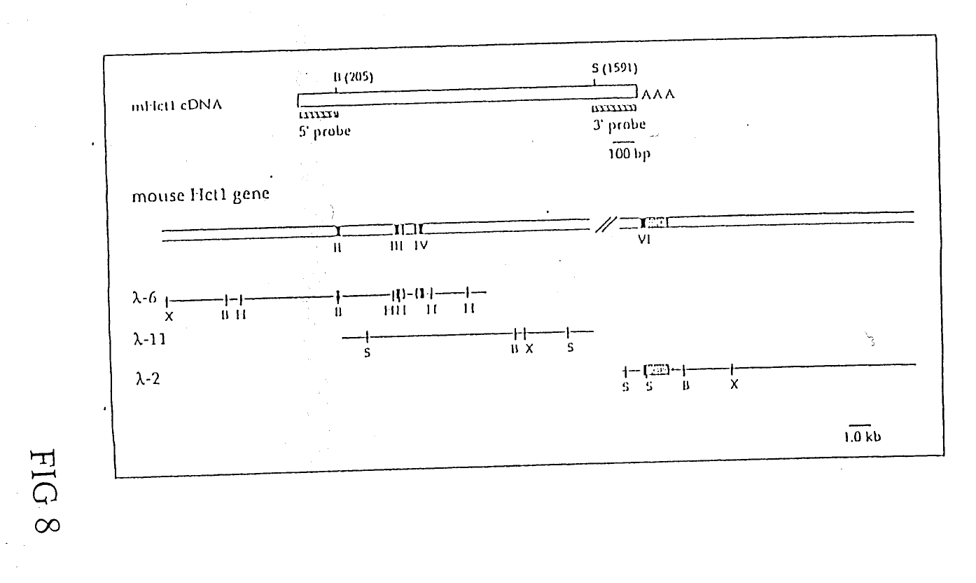

These phage clones were mapped by a combination of restriction enzymes which either cut the lambda clones rarely, or by using restriction sites found in the mHct-1 cDNA sequence (FIG. 3). A 5′ probe was created using a 200 bp fragment from the 5′ end of mHct-1 cDNA as a probe; this segment extended from the internal BamHI site to an, EcoRI site located in the polylinker. The 200 bp 3′ cDNA probe extended from the Sacl site to the polylinker NotI site. Exon-intron boundaries were determined by subcloning of exon-containing genomic DNA fragments and sequencing (FIG. 8).

Phage clones I-6 and I-11 represented 20 kb of contiguous sequence of the Hct-1 locus. I-2 does not overlap withI-6 or I-11, thus the map of the Hct-1 gene in mouse is incomplete. However, the present map shows that mHct-1 spans at least 25 kb of the genome. At least two exons are contained within I-6. The first exon (referred to as exon II) contains 133 bp of coding sequence, followed by exon III, located 4.0 kb downstream. The 3′ boundary of this latter exon is not defined, however approximately 400 bp downstream of its 3′ boundary commences exon IV, which together comprise 797 bp of coding sequence. Exon III and IV are also represented in the overlapping sequence of I-11. A fourth exon of at least 345 bp was identified in I-2 (referred to as exon VI). The 3′ boundary of this exon has not been identified, thus it is not known whether this contains the remaining coding sequence or if there are additional exons.

The following Table provides a summary of the exon-intron structure of Hct-1 (incomplete) and comparison to human CYP7 gene structure. * indicates that these exons are not cloned and are not necessarily one exon. * * indicates that the 3′ boundary of exon VI is not confirmed and may not necessarily be the final exon.

| | cDNA sequence | | |
|---|---|---|---|
| Exon | represented | exon size (bp) | CYP7 exon (bp) |
| I* | 1-142 | 142 | 144 |
| II | 143-275 | 133 | 241 |
| III | 276-? | 797 | 587 |
| IV | ?-1072 | ″ | 131 |
| V* | 1073-1246 | 174 | 176 |
| VI** | 1247-(1821) | (575) | 1596 |

As shown in the Table, cDNA sequence from nucleotides 1073-1246 is not represented in the identified exons and must be represented in a separate exon. 142 bp of 5′ sequence and 227 bp of 3′ sequence have not yet been located in the genomic clones. The remaining 5′ sequence is most likely contained in one exon, as the 5′ probe (BamHI fragment) consistently recognised two bands by Southern analysis (one of which is exon II sequence). The remaining 3′ sequence has not been located and may be part of exon VI or be encoded by a separate exon.

3. Isolation of Human Genomic Sequences for HCT-1

3.1 Conservation of Hct-1 in Humans.

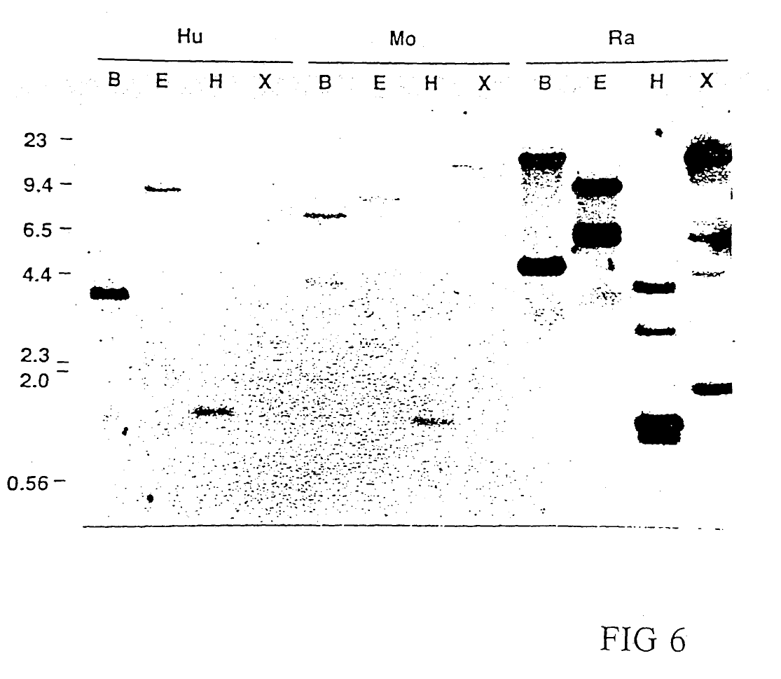

The evolutionary conservation of a gene supports a functionally significant role for that gene in the organism. The conservation of Hct-1 in rodents has been demonstrated by the cloning of the rat and mouse cDNAs for Hct-1. To establish the presence of the Hct-1 gene in the human genome, Southern blotting of human DNA was performed. The rat 1.4 kb clone of Hct-1 was used as a radiolabelled probe and gave strong signals from all three species (FIG. 6). A number of hybridising fragments appear to be conserved between species, suggesting conservation of the Hct-1 gene structure. There is a conserved 1.4 kb Hindlul band between mouse and rat, while human DNA contains a slightly larger HindIII band of 1.6 kb. Also an EcoRI fragment of 11 kb is conserved in human and rat Hct-1. Conservation of Hct-1 gene structure is also supported from the cDNA digestion patterns of mouse and rat (see FIGS. 6 and 7), where the SacI, HindIII and PstI sites are conserved between the rodent species.

3.2 A Single Gene for Hct-1 in Mouse, Rat and Human

Because CYP's comprise a family of related enzymes we wished to determine whether close homologs of Hct-1 are present in the mammalian genome. The rat Hct-1 probe (1.4 kb) was used to probe a genomic Southern blot of rat, mouse and human DNA. In FIG. 6 the probe revealed a simple pattern of cross-hybridizing bands in all DNA's examined. In BamHI-cut human DNA only a single major cross-hybridizing band (4 kb) was detected (FIG. 6), while reprobing with the 300 nt. clone 14-5a yielded, in each lane, a single cross-hybridizing band (not shown). These data argue that a single conserved Hct-1 gene is present in mouse, rat and human, and that the mammalian genome does not contain very close homologs of Hct-1 that would be detected by cross-hybridization (>70-80% homology).

3.3 Isolation of Sequences Encoding Human Hct-1

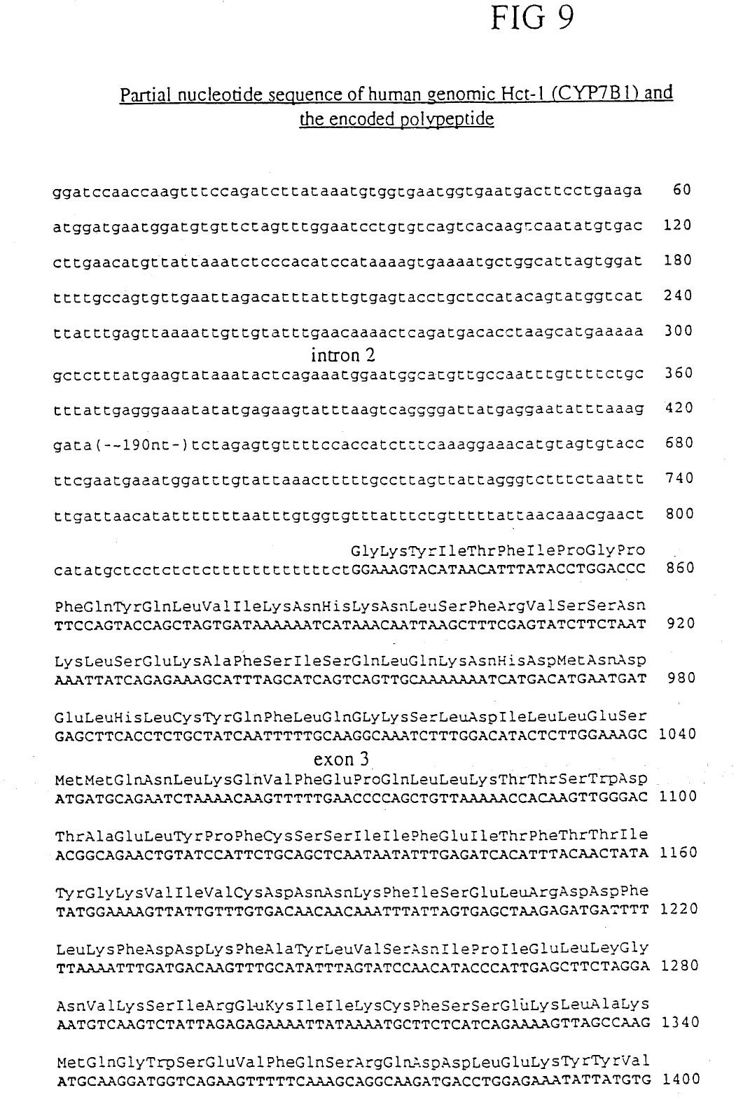

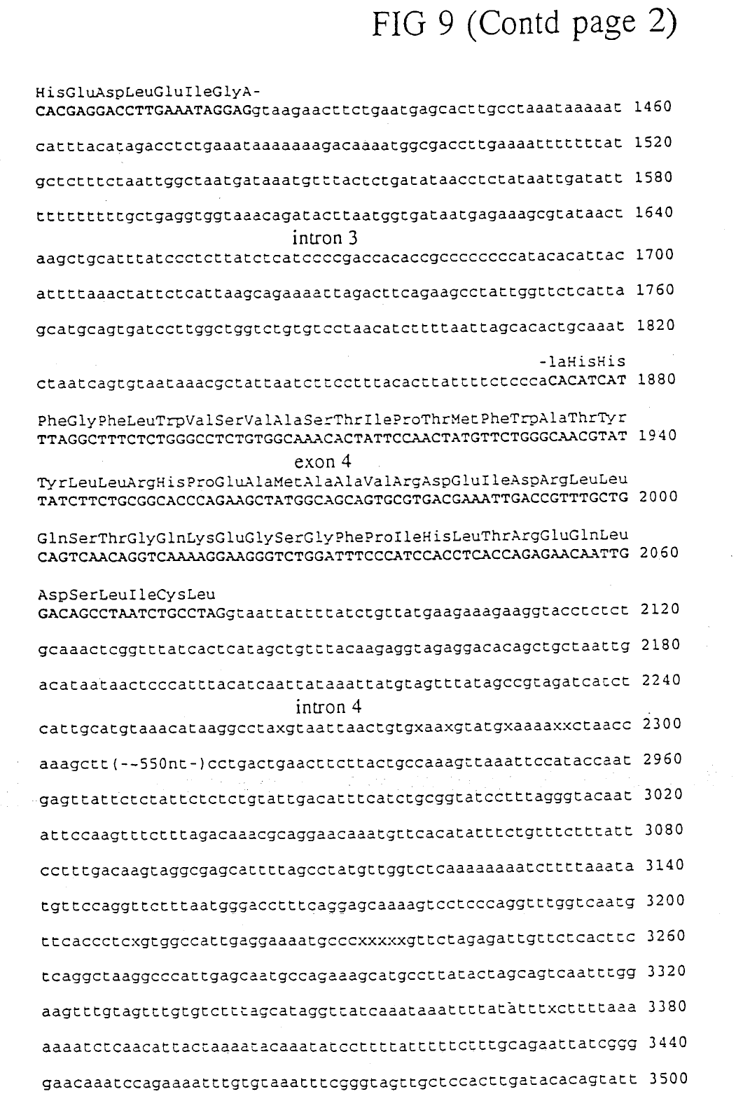

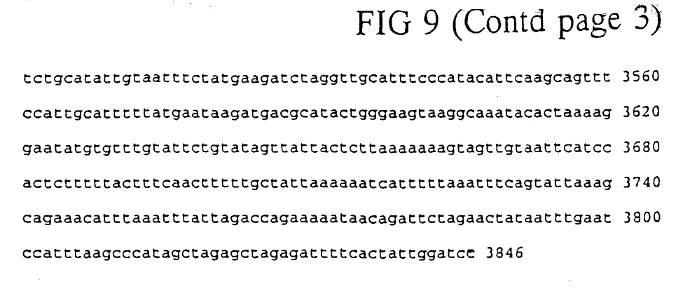

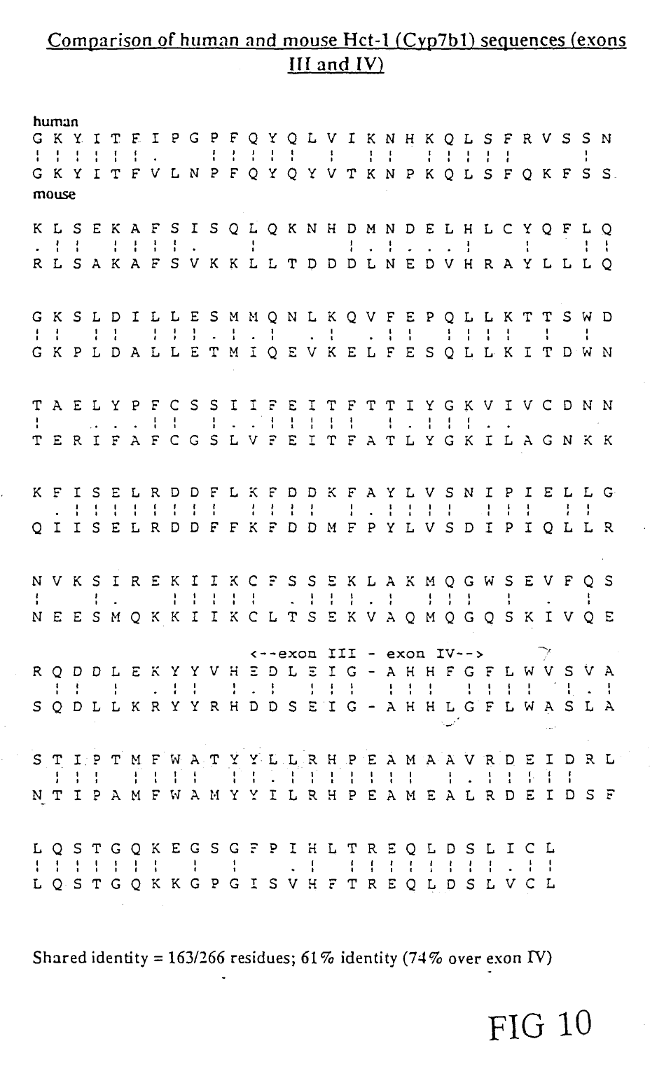

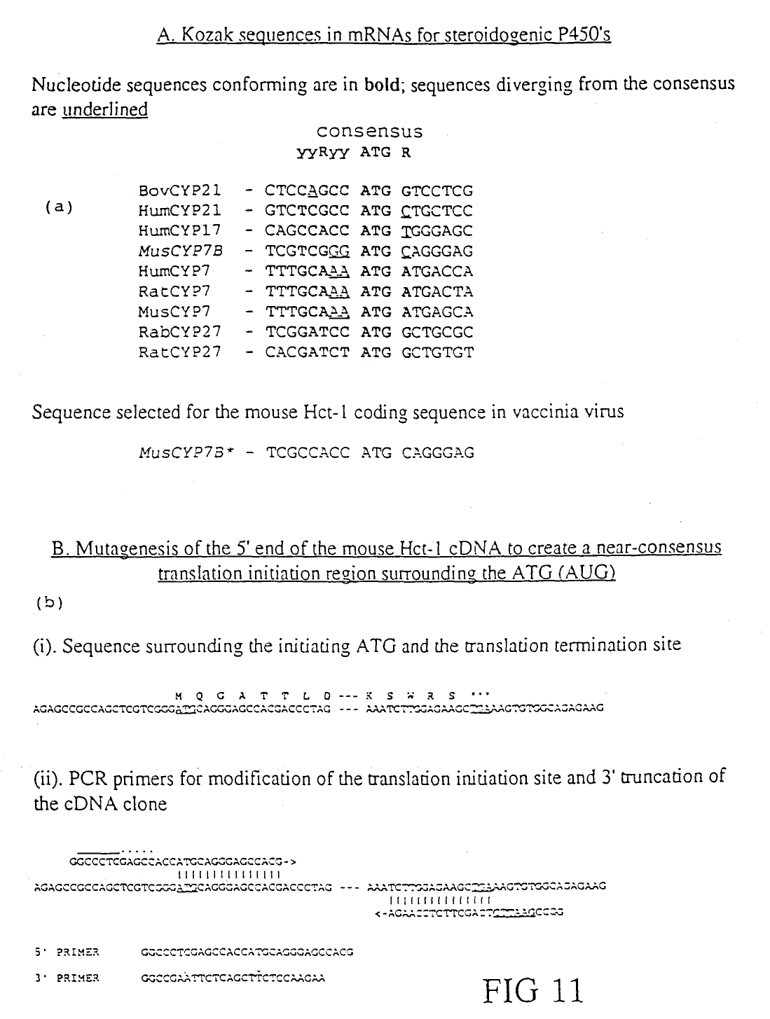

The rat cDNA clone 14.5a-12 was used to probe a Southern blot of human genomic DNA digested with BamHI according to standard procedures. A single band at 3.8 kb was identified that cross-hybridises with the probe. Accordingly, 20 μg of human genomic DNA was cleaved to completion with BamHI, resolved by agarose gel electrophoresis, and the size range 3.4-4.2 kb selected by reference to markers run on the same gel. The gel fragment was digested by agarase treatment, DNA was purified by phenol extraction and ethanol precipitation, and ligated into BamHI-cut bacteriophage lambda ZAP vector (Stratagene). Following packaging in vitro and plating on a lawn of E. coli strain XL1-Blue , plaque lifts of 100,000 clones were screened for hybridisation to the rat cDNA. 12 positive signals were identified and all contained a 3.8 kb insert. One was selected and the segment was partially sequenced, identifying two regions of high homology to the rat (and mouse) cDNA's and corresponding to exons 3 and 4. FIG. 9 presents the nucleotide sequence and FIG. 10 compares the human Hct-1 translation product with the cognate mcuse polypeptide.

To extend this characterisation, the 3.8 kb BamHI fragment obtained from the size-selected library was used to screen a genomic library of human DNA prepared by partial Sau3A cleavage and insertion of 14-18 kb fragments into a bacteriophage lambda vector according to standard techniques (gift of Dr. P. Estibeiro, CGR). Positive clones were obtained, and restriction mapping of one confirmed that it contains approximately 14 kb of human DNA encompassing the exons identified above and further regions of the Hct-1 gene; together the different genomic clones are thought to encompass the entire Hct-1 gene. The human genomic sequence may be used to screen human cDNA libraries for full length cDNA clones; alternatively, following complete DNA sequence determination the human genomic sequence may be expressed in mammalian cells by adjoining it to a suitable promoter sequence and cDNA prepared from the correctly spliced mRNA product so produced. Finally, the genomic Hct-1 sequence would permit the entire coding sequence to be deduced so permitting the assembly of a full length Hct-1 coding sequence by de novo synthesis.

3.4 Expression of Hct-1 Protein for Enzymatic Activity Analysis

3.4.1. Expression of Hct-1 Polypeptide in Yeast Cells

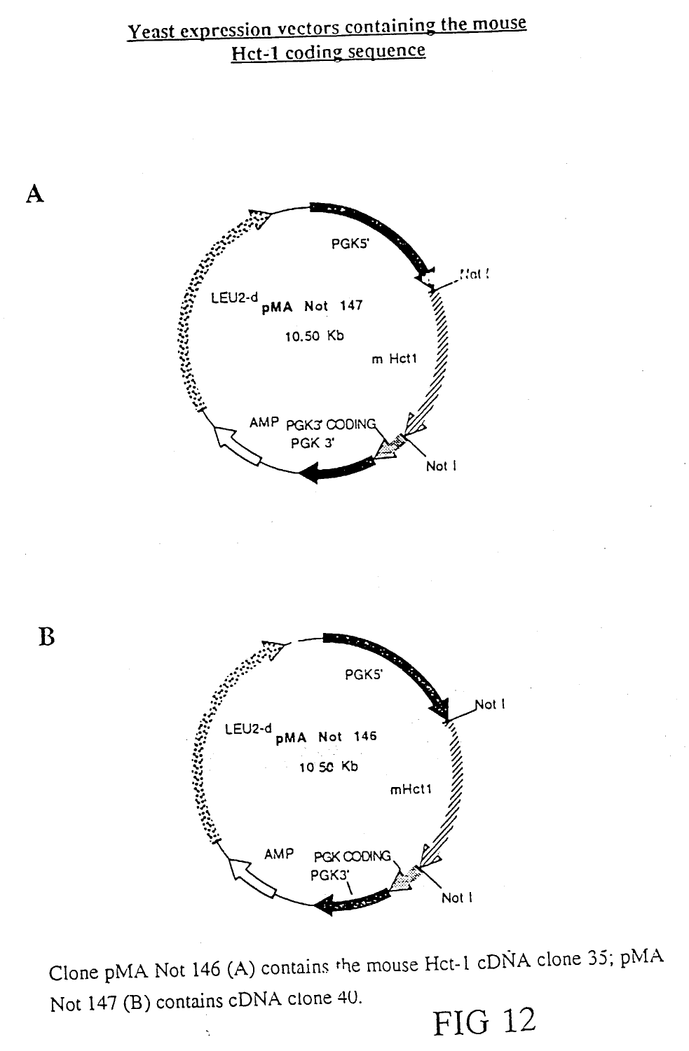

Recombinant yeast strains are useful vehicles for the production of heterologous cytochrome P450 proteins. It would be possible to express any of the mammalian Hct-1's in yeast, but for simplicity we selected the mouse Hct-1 clone 35. To introduce the mouse Hct-1 (mHct-1) coding sequence into yeast the expression vector pMA91 (Kingsman et al., Meth. Enzymol. 185: 329-341, 1990) was employed. The unique Bgll site in pMA91 was converted to a NotI site by inserting the oligonucleotide 5′ GATCGCGGCCGC3′ according to standard procedures. Following cleavage of the resulting plasmid (pMA91 -Not) with NotI the mHct-1 cDNA clone 35 was introduced, placing mHct-1 expression under the control of the yeast PGK (phosphoglycerokinase) promoter for high level expression in yeast cells (FIG. 12A). A similar construct utilising the mHct-1 cDNA clone 35 is depicted in FIG. 12B. Expression of mHct-1 in yeast using these plasmid permits the purification of the protein and determination of substrate specificity.

3.4.2. Expression of Hct-1 Polypeptide in Vaccinia Virus

Expression in vaccinia virus is a routine procedure and has been widely employed for the expression of heterologous cytochromes P450 in mammalian cells, including HepG2 and Hela cells (Gonzalez, Aoyama and Gelboin, Meth. in Enzymol. 206: 85-92, 1991; Waxman et al., Archives Biochem. Biophys 290, 160-166,1991). Accordingly we selected plasmid pTG186-poly (Lathe et al., Nature 326, 878-880, 1987) as the transfer/expression vector, although other similar vectors are widely available and may also be employed.

To demonstrate the expression of mammalian Hct-1's in vaccinia virus, for simplicity we selected the mHct-1 clone 35. Similar techniques are applicable to rat and human Hct-1's. To enhance expression we elected to modify the 5′ end to conform better to the translation consensus for mammalian cells (YYAYYATGR) though this modification may not be essential.

Accordingly, two oligonucleotides were designed corresponding to the 5′ and 3′ regions of the mouse cDNA.

The 5′ oligonucleotide:

(5′-GGCCCTCGAGCCACCATGCAGGGGAGCCACG-3′)

is homologous to the region surrounding the translation initiation site but converts the sequence immediately prior to the ATG to the sequence CCACC; in addition, the oligonucleotide contains a Xhol restriction site for subsequent cloning. The 3′ oligonucleotide (GGCCGAATTCTCAGCTTCTCCAAGAA) was chosen according to the sequence downstream of the translation stop site and contains, in addition, an EcoRI site for subsequent cloning. These oligonucleotides were employed in polymerase chain reaction (PCR) amplification through 5 cycles on the clone 35 template; the products were applied to an agarose gel and the desired product band at 1.65 kb was cut out and extracted by standard procedures.

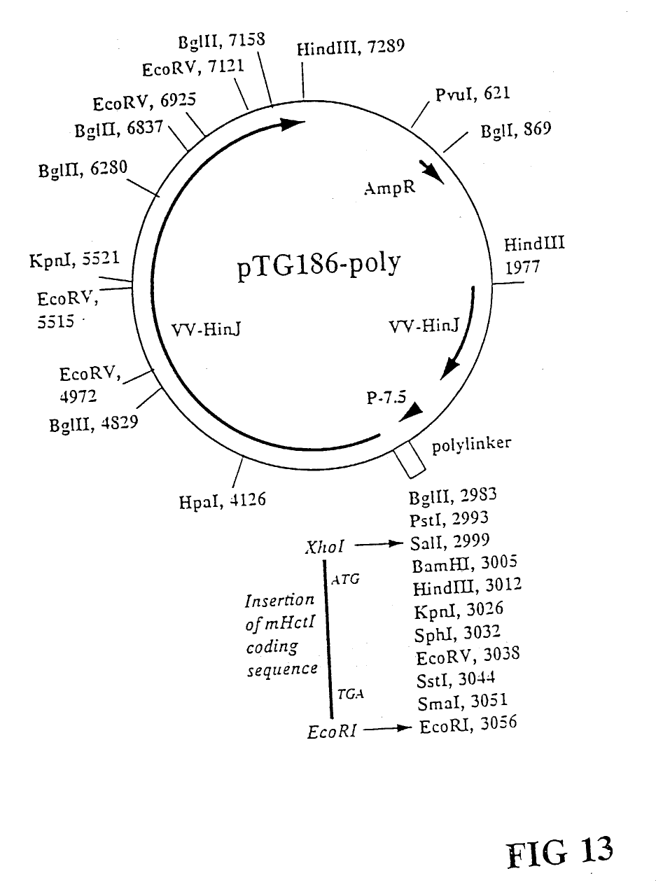

Following cleavage with XhoI and EcoRI the modified fragment was introduced between the EcoRI and SalI sites of pTG186-poly, generating pVV-mHct-1. Recombinational exchange was used to transfer the expression vector to the vaccinia virus genome according to standard procedures, generating VV-mHct-1, as depicted in FIG. 13. This recombinant will permit the expression of high levels of mHct-1 and the identification of the substrate specificity of the protein, as well as the production of antibodies directed against mHct-1.

To identify the product of P450-mediated metabolism, microsomes may easily be prepared (Waxman, Biochem. J. 260: 81-85, 1989) from vaccinia-infected cells: these are incubated with labelled precursors, eg. steroids, and the product identified by thin layer chromatography according to standard procedures (Waxman, Methods in Enzymology 206:462-476).

The Hct-1 provided according to this invention thereby provides a route for the large-scale production of the product described above, for instance a modified steroid, by expressing the P450 in a recombinant organism and supplying the substrate for conversion. It will also be possible to engineer recombinant yeast, for instance, to synthesise the substrate for the Hct-1 P450 in vivo, so as to allow production of the Hct-1 product from yeast supplied with a precursor, for instance cholesterol or other molecule, if that yeast is engineered to contain other P450's or modifying enzymes. It may be possible for Hct-1 to act on endogenous sterols and steroids in yeast to yield product.

Finally, the Hct-1 product may be part of a metabolic chain, and recombinant organisms may be engineered to contain P450's or other enzymes that convert the Hct-1 product to a subsequent product that may in turn be harvested from the organism.

4. Discussion

In experiments to characterize transcripts enriched in the hippocampal formation we isolated cDNA clones corresponding to Hct-1 (hippocampal transcript) from a library prepared from rat hippocampus RNA. In rat, expression appeared to be most abundant in hippocampus with some expression in cortex and substantially less expression other in brain regions. Elsewhere in the body transcripts were only detected in liver and, to a lesser extent, in kidney; expression was barely detectable in ovary, testis and adrenal, also sites of steroid transformations. Hepatic expression was sexually dimorphic with Hct-1 mRNA barely detectable in female liver. In rat brain and liver, Hct-1 identifies two transcripts of 1.8 and 2.1 kb that appear to be generated by alternative polyadenylation; a 5.0 kb transcript weakly detected in brain is thought not to originate from the Hct-1 gene but instead encodes a polypeptide related to the GTPase activating protein, ABR (active BCR-related).

Sequence analysis of Hct-1 cDNA clones revealed an extensive open reading frame encoding a protein with homology to cytochromes P450 (CYP's), a family of heme-containing mono-oxygenases responsible for a variety of steroid and fatty acid interconversions and the oxidative metabolism of xenobiotics. Although the mouse cDNA coding region appears complete, the absence of a consensus translation initiation site flanking the presumed initiation codon could indicate that Hct-1 polypeptide synthesis is subject to regulation at the level of translation initiation.

Homology was highest with rat and human cholesterol 7α-hydroxylase, known as CYP7. While related, Hct-1 is clearly distinct from CYP7, sharing only 39% homology over the full length of the protein. CYP polypeptides sharing greater than 40% sequence identity are generally regarded as belonging to the to the same family, and Hct-1 and CYP7 (39% similarity) are hence borderline. The conservation of other unique features between Hct-1 and CYP7 however argues for a close relationship and Hct-1 has been provisionally named ‘CYP7B’ by the P450 Nomenclature Committee (D. R. Nelson, personal communication).

From the Hct-1 leader sequence we surmise that the Hct-1 polypeptide resides, like CYP7, in the endoplasmic reticulum and not in mitochondria, the other principal cellular site of CYP activity. The strictly conserved heme binding site motif FxxGxxxCxG(xxxA) is clearly present in Hct-1 (residues 440-453). It is of note that the ‘steroidogenic domain’, conserved in many CYP's responsible for steroid interconversions, is also present in Hct-1 (amino acids 348-362), except that a consensus Pro residue is replaced by Val in both the mouse and rat Hct-1 polypeptides. Of previously known 34 CYP sequences, only 4 contain an amino acid residue other than Pro at this position. Whereas 2 of these harbour an unrelated amino acid (Glu; CYP3A1, CYP3A3), interestingly, a Val residue is present in bovine CYP17 (steroid 17α-hydroxylase, 44) at a position equivalent to that in Hct-1 while human CYP17 harbours a conservative substitution at this site (Leu; 44). Despite this similarity, however, the overall extent of homology between Hct-1 and CYP17 (22.5%, not shown) is lower than with CYP7 (39%)

Neither Hct-1 and CYP7 appear to contain a conserved O₂ binding pocket (equivalent to residues 285-301 in Hct-1). Crystallographic studies on the bacterial CYP101 indicated that a Thr residue (corresponding to position 294 in Hct-1) disrupts helix formation in that region and is important in providing a structural pocket for an oxygen molecule. Site-directed mutagenesis of this Thr residue in both CYP4A1 and CYP2C11 demonstrated that this region can influence substrate specificity and affinity. In both Hct-1 and CYP7 the conserved Thr residue is replaced by Asn. This modification suggests that Hct-1 and CYP7 are both structurally distinct from other CYP's in this region; this may be reflected both in modified oxygen interaction and substrate choice.

The sexual dimorphism of Hct-1 expression observed in rat resembles that observed with a number of other CYP's. CYP2C12 is expressed preferentially in liver of the female rat while, like Hct-1, CYP2C11 is highly expressed in male liver but only at low levels in the female tissue. This dimorphic expression pattern of CYP2C family members is thought to be determined by the dimorphism of pulsatility of growth hormone secretion. Brain expression of Hct-1 is not subject to this control suggesting that regulatory elements determining Hct-1 expression in brain differ from those utilized in liver. However, we have not examined species other than rat; it cannot be assumed that the same regulation will exist in other species. Indeed, sexually dimorphic gene expression is not necessarily conserved between different strains of mouse.

Expression of Hct-1 was widespread in mouse brain. The expression pattern was most consistent with glial expression but further experiments will be required to compare neuronal and non-neuronal levels of expression. In mouse brain only the 1.8 kb transcript was detected, though cDNA's were obtained corresponding to transcripts extending beyond the first polyadenylation site; such extended transcripts are thought to give rise to the 2.1 kb transcript in rat. This suggests the downstream polyadenylation site seen in rat Hct-1 is under-utilized in mouse Hct-1 or absent. While in situ hybridization studies of Hct-1 in rat brain were inconclusive, a difference in expression pattern between mouse and rat appears likely; further work will be required to confirm this. However, such a difference would be unsurprising because cytochromes P450 are well known to vary widely in their level and pattern of expression in different species; for instance, hepatic testosterone 16-hydroxylation levels differ by more than 100-fold between guinea pig and rat.

Our data indicate that the Hct-1 gene is present in rat, mouse and human, and there appear to be no very close relatives in the mammalian genome. While CYP genes are scattered over the mouse and human genomes, CYP subfamilies can cluster on the same chromosome. For instance, the human CYP2A and 2B subfamily genes are linked to chromosome 19, CYP2C and 2E subfamilies are located on human chromosome 10, and the mouse cyp2a, 2b and 2e subfamilies are present on mouse chromosome 7. The gene encoding human cholesterol 7α-hydroxylase (CYP7) is located on chromosome 8q 11 -q 12.

Together our data argue that Hct-1 and CYP7 are closely related: this suggests that the substrate for Hct-1, so far unknown, is likely to be related to cholesterol or one of its steroid metabolites. This interpretation is borne out by the presence, in Hct-1, of the steriodogenic domain conserved in a number of steroid-metabolizing CYP's. While experiments are underway to determine the substrate specificity of Hct-1, the possibility that Hct-1 acts on cholesterol or its steroid metabolites in brain is of some interest. CYP7 (cholesterol 7α-hydroxylase) is responsible for the first step in the metabolic degradation of cholesterol. This is of note in view of the association of particular alleles of the APOE gene encoding the cholesterol transporter protein apoiipoprotein E with the onset of Alzheimer's disease, a neurodegenerative condition whose cognitive impairments are associated with early dysfunction of the hippocampus.

What role might Hct-1 play in the brain? In the adult CYP's are generally expressed abundantly in liver, adrenal and gonads, while the level of CYP activity in brain is estimated to be 0.3 to 3% of that found in liver (see 58). Because levels of Hct-1 mRNA expression in rat and mouse brain far exceed those in liver it could be argued that the primary function of Hct-1 lies in the central nervous system. The documented ability of cholesterol-derived steroids to interact with neurotransmitter receptors and modulate both synaptic plasticity and cognitive function suggests that Hct-1 and its metabolic product(s) may regulate neuronal function in vivo.

5. Summary

Hct-1 (hippocampal transcript) was detected in a differential screen of a rat hippocampal cDNA library. Expression of Hct-1 was enriched in the formation but was also detected in rat liver and kidney, though at much lower levels; expression was barely detectable in testis, ovary and adrenal. In liver, unlike brain, expression was sexually dimorphic: hepatic expression was greatly reduced in female rats. In mouse, brain expression in was widespread, with the highest levels being detected in corpus callosum; only low levels were detected in liver. Sequence analysis of rat and mouse Hct-1 cDNAs revealed extensive homologies with cytochrome P450's (CYP's), a diverse family of heme-binding monooxygenases that metabolize a range of substrates including steroids, fatty acids and xenobiotics. Among the CYP's, Hct-1 is most similar (39% at the amino acid sequence) to cholesterol 7α-hydroxylase (CYP7), and contains the diagnostic steriodogenic domain present in other steriod-metabolizing CYPs, but clearly represents a type of CYP not previously reported. Genomic Southern analysis indicates that a single gene corresponding to Hct-1 is present in mouse, rat and human. Hct-1 is unusual in that, unlike all other CYP's described, the primary site of expression is in the brain. Similarity to CYP7 and other steroid-metabolizing CYP's argues that Hct-1 plays a role in steroid metabolism in brain, notable because of the documented ability of brain-derived steroids (neurosteroids) to modulate cognitive function in vivo.

6. Details of Experimental Protocols

Northern analysis—Total RNA was extracted by tissue homogenization in guanidinium thiocyanate according to a standard procedure and further purified by centrifugation through a CsCl cushion. Where appropriate, polyA-plus RNA was selected on oligo-dt cellulose. Electrophoresis of RNA (10 μg) on 1% agarose in the presence of 7% formaldehyde was followed by capillary transfer to nylon membranes, baking (2 h, 80° C.), and rinsing in hybridization buffer (0.25 M NaPhosphate, pH 7.2; 1 mM EDTA, 7% sodium dodecyl sulphate [SDS], 1% bovine serum albumin) as described (Church et al., supra). Probes were prepared by random-priming of DNA polymerase copying of denatured double-stranded DNA. Hybridization (16 h, 68° C.) was followed by washing (3 times, 20 mM NaPhosphate pH 7.2, 1 mM EDTA, 1% SDS, 20 min.) and membranes exposed for autoradiography. The loading control probe was a 0.5 kb cDNA encoding the ubiquitously expressed rat ribosomal protein S26.

In situ Hybridization—Synthetic Hct-1 Oligonucleotide Probes

5′-dGACAGGTTTTGTGACCCAAAACAAACTGGATGGATCGCAATC-3′
(rat, 55% G + C) and
5′-ATCACGGAGCTCAGCACATGCAGCCTTACTCTGCAAAGCTTC3′
(mouse-48% G = C)

(mouse−48% G+C) were labelled using terminal transferase (Boehringer Mannheim) and α-³⁵S-dATP (Amersham) according to the manufacturer's instructions.

The control probe, 5′-dAGCCTTCTGGGTCGTAGCTGACTCCTGCTGCTGAGCTGCAACAGCTTT-3′ (56% G+C) was based on human opsin cDNA. Frozen coronal 10 μm sections of brain were fixed (4% paraformaldehyde, 10 min), rinsed, treated with proteinase K (20 μg/ml in 50 mM Tris.HCl, pH 7.4, 5 mM EDTA, 5 min), rinsed, and refixed with paraformaldehyde as before. Following acetylation (0.25% acetic anhydride, 10 min) and rinsing, sections were dehydrated by passing though increasing ethanol concentrations (30, 50, 70, 85, 95, 100, 100%, each for 1 minute except the 70% step [5 min]). Following CHCl₃ treatment (5 min), and rinsing in ethanol, sections were dried before hybridization. Hybridization in buffer (4× standard saline citrate [1×SSC=0.15 M NaCl, 0.015 M Na₃citrate], 50% v/v formamide, 10% w/v dextran sulphate, 1× Denhardt's solution, 0.1% SDS, 500 μg/ml denatured salmon sperm DNA, 250 μg/ml yeast tRNA) was for 16 h at 37° C. Slides were washed (4×15 min., 1×SSC, 60° C.; 2×30 min., 1×SSC, 20° C.), dipped into photographic liquid emulsion (LM-1, Amersham), exposed and developed according to the manufacturer's specifications. Slides were counterstained with 1% methyl green.

Southern hybridization—Genomic DNA prepared from mouse or rat liver, or from human lymphocytes, was digested with the appropriate restriction endonuclease, resolved by agarose gel electrophoresis (0.7%) and transferred to Hybond-N membranes. Following baking (2 h, 80° C.), hybridization conditions were as described for Northern analysis.

Hybridisation Conditions. Hybridisation conditions used were based on those described by Church and Gilbert, Proc. Natl. Acad. Scl. USA (1984) 81, 1991-1995.

1. Filters were pre-wet in 2×SSC.

2. The hybridisation was performed in a rotating glass cylinder (Techne Hybridiser ovens). 10 ml of Hybridisation Buffer was added to the cylinder with the filter.

3. Prehybridisation and hybridisation were carried out at 68° C. unless otherwise specified.

4. The filters were prehybridised for 30 minutes, after which the probe was added directly and hybridisation proceeded overnight. (Double-stranded probes were denatured by boiling for 2 minutes, then placing on ice).

5. Washes were performed at 68° C. (unless otherwise stated) with 2 changes of Wash Buffer I for 10 minutes each, followed by three changes of Wash Buffer II each for 20 minutes.

6. The filters were blotted dry, but not allowed to dry out, then placed between Saran wrap, and against X-ray film for autoradiography.

Hybridisation Buffer:

0.25 M sodium phosphate pH 7.2

1 mM EDTA

7% SDS

1% BSA

Wash Buffer I:

20 mM sodium phosphate pH 7.2

2.5% SDS

0.25% BSA

1 mM EDTA

Wash Buffer II:

20 mM sodium phosphate pH 7.2

1 mM EDTA

1% SDS

Screening of Bacteriophage lambda libraries. The rat hippocampus cDNA library was oligo-(dT)-NotI primed and cloned in lambda ZAP II (Stratagene) with an EcoRI adaptor at the 5′ end, and was prepared in the lab by Miss M. Richardson and Dr. J. Mason; the mouse liver cDNA library was oligo-(dT)-primed and cloned into lambda gt10 with EcoRI/NotI adaptors, and was a gift from Dr. B. Luckow, Heidelberg; the mouse ES cell genomic library was cloned from a partial Sau3A digest into lambda DASH II (Stratagene), and was a gift from A. Reaume, Toronto.

The libraries were screened as described above by hybridization.

In vivo excision of pBluescript from lambda ZAP II vector was performed using the ExAssist/SOLR system (Stratagene, 200253).

In situ hybridisation. Frozen 10μ coronal sections of rat and mouse brains were provided by Dr. M. Steel.

Hybridisation Conditions All probes were oligonucleotides which were labelled by homopolymer tailing using a-³⁵S-dATP and terminal transferase.

The sequences or references of the oligonucleotides used as probes for in situ hybridisation were as follows:

rat Hct-1 (a 45-mer, beginning 26 nt 5′ from the polyA tail, nucleotides 1361-1403 in FIG. 4.2) (for relative position in mouse gene, see FIG. 4.3) 5′-GACAGGTTTTGTGACCCAAAACAAACTGGATGGATCGCAATC-3′

Nathans mouse Hct-1 (nt 1558-1599) 5′-ATCACGGAGCTCAGCACATGCAGCCTTACTCTGCAAAGCTTC-3′

rat clone 13 (a 42-mer, beginning 112 nt 5′ from polyA tail) 5′-TATATCCATACCAACTTATTGGGAGTCCCATCCTACCTCATCAGC-3′

rat/mouse muscarinic receptor M1 (Buckley et al., 1988)

rat/mouse opsins (Nathans et al., Science (1986) 232, 193-202)

1. The prepared ³⁵S-tailed probe (resuspended in 10 mM DTT in TE) was diluted to 2×10⁶ cpm/ml in hybridisation buffer. DTT is also added to this mixture to a final concentration of 50 mM.

2. 100 ml of the probe mixture was carefully layered onto each microscope slide. A piece of parafilm cut to the size of the microscope slide was then layered over the probe mixture, allowing the probe and hybridisation mixture to cover all the sections. Air bubbles under the parafilm were avoided.

3. The slides were placed in a humidified container, sealed, and incubated at 37° C. overnight.

4. After hybridisation, the parafilm was carefully removed using forceps.

5. The slides were placed back in Coplin jars, and the hybridised sections washed in four changes of 1×SSC for 15 minutes at 55° C. or 60° C., and then two changes of 1×SSC for 30 minutes at room temperature.

6. The slides were rinsed briefly in dH₂O, then left to air dry.

Hybridisation Buffer*:

4×SSC

50% (v/v) deionised formamide

10% (w/v) dextran sulphate

1× Denhardt's solution

0.1% (w/v) SDS

500 μg/ml ssDNA

250 μg/ml yeast tRNA

*buffer was de-gassed before use

7. Figure Legends

FIG. 1. Sequence of partial rat Hct-1 cDNA and the encoded polypeptide. The nucleotide sequence and translation product of the 1.4 kb cDNA clone 12 including additional clone 7 sequence (lower case). The two putative polyadenylation signals are underlined.

FIG. 2. Northern analysis of Hct-1 expression in adult rat and mouse brain. Panel A. Expression in rat brain and other tissues; panel B. sexually dimorphic expression in rat liver; panel C. Expression in mouse tissues. Poly-A⁺ (A) or total B,C) RNA from organs of adult animals were resolved by gel electrophoresis; the hybridization probe was rat Hct-1 cDNA clone 12 (1.4 kb), the probe for the loading control (below) corresponds to ribosomal protein S26. Tissues analysed are: Hi, hippocampus; RB, remainder of brain lacking hippocampus; Cx, cortex; Cb, cerebellum; Ob; olfactory bulb; Li, liver; He, heart; Th, thymus; Ki, kidney; Ov, ovary; Te, testis; Lu, lung.

FIG. 3. Mouse Hct-1 cDNA and the sequence of the encoded polypeptide. The restriction map of the cDNA (above) corresponds to the compilation of two independent clones sequenced; the cross-hatched box indicates the coding region. The nucleotide sequence and translation product (below) derives from this compilation. Lower case sequences indicate the 59 additional 5′ nucleotides in clone 40 and the 99 additional 3′ nucleotides in clone 35. The putative polyadenylation site is underlined.

FIG. 4. Alignment of mouse Hct-1 with human CYP7 (cholesterol 7α-hydroxylase, Noshiro and Okuda, 1990) and other steroidogenic P450s. Panel A: Identical amino acids are indicated by a bar; hyphens in the amino acid sequences indicate gaps introduced during alignment. The N-terminal hydrophobic leader sequences are underlined. The position of the conserved Thr residue within the O₂-binding pocket of other CYP's (43), but replaced by Asn in Hct-1 (position 294) and CYP7, is indicated by an asterisk. Panels B,C: conserved residues in the heme-binding (residues 440-453, B) and steroidogenic (residues 348-362, C) domains conserved between Hct-1 and other similar CYP's (overlined in A). Sequences are human CYP7 (7α-hydroxylase; 37); bovine CYP17 (17α-hydroxylase; 44); human CYP11B1 (steroid β-hydroxylase; 45); human CYP21B (21-hydroxylase; 11); human CYP11A1 (P450scc; cholesterol side-chain cleavage; 46); human CYP27 (27-hydroxylase; 47).

FIG. 5. Analysis of Hct-1 expression in adult mouse brain. The hybridization probe was a synthetic oligonucleotide corresponding to the 3′ untranslated region of mouse Hct-1 cDNA. Panel a: coronal section; panel b: coronal section, rostral to a, showing hybridization in corpus callosum, cc; fornix, f; and anterior commissure, ac; panel c: enlargement of section through the hippocampus; DG, dentate gyrus; panel d: section adjacent to the section in a hybridized with an oligonucleotide specific for opsin (negative control).

FIG. 6. Southern analysis of Hct-1 coding sequences in mouse, rat and human Total DNA was cleaved as indicated with restriction endonucleases B, BamHI; E, EcoRI; H, HindIII; X, XbaI; resolved by agarose gel electrophoresis, and probed with rat Hct-1 cDNA clone 12 before exposure to autoradiography.

FIG. 7 Genomic DNA Southern blot analysis of Hct-1 (a) Mouse genomic DNA probed with the full-length mouse Hct-1 cDNA clone. (b) Rat genomic DNA probed with clone 14.5a (original 0.3 kb clone of rHct-1). 10 μg of genomic DNA was digested with the indicated enzymes.

FIG. 8 Genomic map of mouse Hct-1 (incomplete). Exons II, III, IV and VI are represented on the phage clones (filled boxes). Exons I and V are not located. As indicated in Table 4.1, the boundaries of exons II, III B (BamHI); H(HindIII); S(SacI); X(XhoI)

1. A DNA molecule selected from the following:
(a) DNA molecules containing the coding sequence set forth in SEQ Id No: 1 beginning at nucleotide 22 and ending at nucleotide 1541,
(b) DNA molecules containing the coding sequence set forth in SEQ Id No: 2 beginning at nucleotide 1 and ending at nucleotide 1242,
(c) DNA molecules capable of hybridizing with the DNA molecule defined in (a) or (b) under standard hybridization conditions defined as 2×SSC at 65° C.
(d) cytochrome P450-encoding DNA molecules capable of hybridizing with the DNA molecule defined in (a), (b) or (c) under reduced stringency hybridization conditions defined as 6×SSC at 55° C.

2. A DNA molecule according to claim 1 (c) or (d) comprising an Hct-1 gene-associated sequence of another vertebrate species, especially a mammalian species and in particular a human Hct-1 gene-associated sequence,

3. A DNA molecule according to claim 2 selected from the following:
(e) DNA molecules comprising one or more sequences selected from
(i) the sequence designated “intron 2” in SEQ Id No 3,
(ii) the sequence designated “exon 3” in SEQ Id No 3,
(iii) the sequence designated “intron 3” in SEQ Id No 3,
(iv) the sequence designated “exon 4” in SEQ Id No 3, and
(v) the sequence designated “intron 5” in SEQ Id No 3; and
(f) DNA molecules capable of hybridizing with the DNA molecules defined in (e) under standard hybridization conditions defined as 2×SSC at 65° C.
(g) cytochrome P450-encoding DNA molecules capable of hybridizing with the DNA molecule defined in (e) or (f) under reduced stringency hybridization conditions defined as 6×SSC at 55° C.

4. A DNA molecule comprising a human Hct-1 gene-associated sequence and selected from the following:
(e) DNA molecules comprising one or more sequences selected from
(i) the sequence designated “intron 2” in SEQ Id No 3,
(ii) the sequence designated “exon 3” in SEQ Id No 3,
(iii) the sequence designated “intron 3” in SEQ Id No 3,
(iv) the sequence designated “exon 4” in SEQ Id No 3, and
(v) the sequence designated “intron 5” in SEQ Id No 3; and
(f) DNA molecules capable of hybridizing with the DNA molecules defined in (e) under standard hybridization conditions defined as 2×SSC at 65° C.
(g) cytochrome P450-encoding DNA molecules capable of hybridizing with the DNA molecule defined in (e) or (f) under reduced stringency hybridization conditions defined as 6×SSC at 55° C.

5. A DNA molecule comprising a human Hct-1 gene-associated sequence and selected from the following:
(h) DNA molecules comprising contiguous pairs of sequences selected from
(i) the sequence designated “intron 2” in SEQ Id No 3,
(ii) the sequence designated “exon 3” in SEQ Id No 3,
(iii) the sequence designated “intron 3” in SEQ Id No 3,
(iv) the sequence designated “exon 4” in SEQ Id No 3, and
(v) the sequence designated “intron 5” in SEQ Id No 3; and
(i) DNA molecules capable of hybridizing with the DNA molecules defined in (h) under standard hybridization conditions defined as 2×SSC at 65° C.
(j) cytochrome P450-encoding DNA molecules capable of hybridizing with the DNA molecule defined in (h) or (i) under reduced stringency hybridization conditions defined as 6×SSC at 55° C.

6. A DNA molecule comprising a human Hct-1 gene-associated coding sequence selected from the following:
(k) DNA molecules comprising a contiguous coding sequence consisting of the sequences “exon 3” and “exon 4” in SEQ Id No 3, and
(l) DNA molecules capable of hybridizing with the DNA molecules defined in (k) under standard hybridization conditions defined as 2×SSC at 65° C.
(m) cytochrome P450-encoding DNA molecules capable of hybridizing with the DNA molecule defined in (k) or (l) under reduced stringency hybridization conditions defined as 6×SSC at 55° C.

7. A DNA molecule encoding an Hct-1 gene-associated coding sequence coded for by a DNA molecule as claimed in any of claims 1 to 6, but which differs in sequence from the sequences of the DNA molecules claimed in claims 1 to 6 by virtue of one or more amino acids of said Hct-1 gene-associated sequences being encoded by degenerate codons.

8. A DNA molecule consisting of a contiguous sequence of at least 1-8 nucleotides from the DNA sequence set forth in SEQ Id Nos: 1, 2 and 3.

9. A DNA sequence according to claim 8 containing at least 24 and most preferably at least 30 nucleotide taken from said sequence.

10. The use of a DNA molecule according to claim 8 or claim 9 as a hybridization probe for isolating or detecting members of gene families and homologous DNA sequences related to the Hct-1 gene, especially a human gene sequence.

11. The use of a DNA molecule according to claim 8 or claim 9 in the diagnosis of neuropsychiatric disorders, endocrine disorders, immunological disorders, diseases of cognitive function or neurodegenerative diseases.

12. The use of a short (e.g. 10 to 25) oligonucleotide primer, capable of hybridising with a DNA molecule claimed in any of claims 1 to 9 in the polymerase chain reaction (PCR) amplification of a genomic or cDNA from a biological sample for the purpose of diagnosis of neuropsychiatric disorders, endocrine disorders, immunological disorders, diseases of cognitive function or neurodegenerative diseases.

13. A cytochrome P450 protein, at leas. a portion of which is encoded by a DNA molecule as claimed in any of claims 1 to 7.

14. A protein selected from the following:
(i) the protein designated rat Hct-1 comprising the amino acid sequence set forth in SEQ Id No: 1 or a protein having substantial homology thereto,
(ii) the protein designated mouse Hct-1 comprising the amino acid sequence set forth in SEQ Id No: 2 or a protein having substantial homology thereto, or
(iii) the protein designated human Hct-1 comprising the amino acid sequence set forth in SEQ Id No: 3: or a protein having substantial homology thereto.

15. A protein according to claim 14 having a degree of homology such that at least 50%, preferably at least 60% and most preferably at least 70% of the amino acids match said Seq.ID No: 1, 2 or 3).

16. A process for producing a Hct-1 polypeptide, which comprises culturing a transformed host and recovering the desired Hct-1 polypeptide, characterised in that the host is transformed with nucleic acid comprising a coding sequence as defined in any of claims 1 to 7.

17. A process according to claim 15 wherein the transformed host cell is a yeast, bacterial, insect or mammalian cell.

18. A process according to claim 16 or claim 17 wherein the nucleic acid comprises an expression construct or an expression vector.

19. A process according to claim 18 wherein the vector is a vaccinia virus or baculovirus vector, a yeast plasmid or integration vector.

20. An antibody, especially a monoclonal antibody which binds to a Hct-1-protein.

21. The use of an antibody according to claim 19 in the diagnosis of neuropsychiatric disorders, endocrine disorders, immunological disorders, diseases of cognitive function, neurodegenerative diseases or diseases of cognitive function.

22. The use of a protein according to any of claims 13 to 15, or an antibody according to claim 18 in the design and/or manufacture of an antagonist to Hct-1 protein.

23. The use of a protein according to any of claims 13 to 15, to effect a catalytic transformation of a substrate.

24. The use according to claim 23 wherein the substrate is a steroid.

25. A transformed substrate when produced as a result of the use claimed in claim 23 or claim 24.
